(19) United States
(12) Patent Application Publication
(10) Pub. No.: US 2023/0371899 A1
(43) Pub. Date: Nov. 23, 2023

(54) BIOLOGICAL INFORMATION MEASURING BELT FOR CALF

(71) Applicant: TOYOBO CO., LTD., Osaka (JP)

(72) Inventors: Tomoyuki MIYAMOTO, Osaka (JP); Yuichiro OMOTE, Osaka (JP); Yoko KOMATSU, Shiga (JP)

(73) Assignee: TOYOBO CO., LTD., Osaka (JP)

(21) Appl. No.: 18/030,546

(22) PCT Filed: Sep. 7, 2021
(86) PCT No.: PCT/JP2021/032867

§ 371 Date: Apr. 6, 2023

(30) Foreign Application Priority Data
Oct. 12, 2020 (JP) 2020-172036

(51) Int. Cl. A61B 5/00

(52) U.S. Cl. CPC A61B 5/6831; A61B 2503/40

(57) ABSTRACT

An object of the present invention is to provide a biological information measuring belt for a calf that facilitates measurement of biological information. The biological information measuring belt including:
a first fabric; and
an electrode provided on a skin-side surface of the first fabric,
wherein a skin-side surface of the biological information measuring belt includes a high friction part having a mean friction coefficient MIU of 0.40 or more,
the biological information measuring belt has a tensile strength of 2.8 N/cm or less at 5% stretching and has a tensile strength of 4.0 N/cm or less at 10% stretching under following conditions, and
the conditions are that the biological information measuring belt is held with chucks of a tensile tester in such a manner that a distance between the chucks becomes 45 cm and a center of the first fabric is positioned at a center between the chucks, and then the biological information measuring belt is stretched at a stretching speed of 100 mm/minute.

### TECHNICAL FIELD

The present invention relates to a biological information measuring belt for a calf.

### BACKGROUND ART

In recent years, biological information measuring garments have drawn attention in a health monitoring field, a medical field, a medical treatment and education field, and a rehabilitation field. For example, Patent Document 1 discloses identifying a measurement position allowing stable measurement of biological information and proposes a sensing wear that has a highly close-attachment flexible electrode mounted thereto.

### RELATED ART DOCUMENTS

### Patent Documents

Patent Document 1: JP2017-29692A

### SUMMARY OF THE INVENTION

### Technical Problem

As seen in Patent Document 1, biological information measuring garments worn by humans are being variously developed. However, in the livestock and dairy industry where health management of cattle is important, a biological information measuring garment for cattle has not been sufficiently developed. Particularly, calves easily become ill and thus need health management, but a belt suitable for measuring biological information of the calves has yet to be developed. The present invention has been made under the above circumstances, and an object of the present invention is to provide a biological information measuring belt for a calf that facilitates measurement of biological information.

### Solutions to the Problems

A biological information measuring belt for a calf according to an embodiment of the present invention that has overcome the above problems is as per [1] below.
[1] A biological information measuring belt for a calf, the biological information measuring belt including:
a first fabric; and
an electrode provided on a skin-side surface of the first fabric,
wherein a skin-side surface of the biological information measuring belt includes a high friction part having a mean friction coefficient MIU of 0.40 or more,
the biological information measuring belt has a tensile strength of 2.8 N/cm or less at 5% stretching and has a tensile strength of 4.0 N/cm or less at 10% stretching under following conditions, and
the conditions are that the biological information measuring belt is held with chucks of a tensile tester in such a manner that a distance between the chucks becomes 45 cm and a center of the first fabric is positioned at a center between the chucks, and then the biological information measuring belt is stretched at a stretching speed of 100 mm/minute.

As described above, the skin-side surface of the biological information measuring belt having the high friction part having the mean friction coefficient MIU of 0.40 or more makes the biological information measuring belt less likely to be positionally displaced, thus being able to facilitate measurement of biological information. Although a calf rapidly grows and becomes large in body peripheral length on a daily basis, the tensile strengths of the biological information measuring belt within the above ranges enable the belt to be stretched in line with the growth of the calf. As a result, a skin abnormality or the like of the calf that is caused by a pressure of the belt can be reduced.

A preferable aspect of the biological information measuring belt is any one of following [2] to [33].
[2] The biological information measuring belt according to above [1], including a band member provided at at least one end part of the first fabric in a body peripheral direction.
[3] The biological information measuring belt according to above [2], including a second fabric provided at at least one end part of the first fabric in the body peripheral direction and/or on the band member in such a manner that the second fabric is positioned closer to skin than the band member.
[4] The biological information measuring belt according to above [2] or [3].
wherein a skin-side surface of the band member includes the high friction part, and
the skin-side surface of the first fabric includes a low friction part having a lower mean friction coefficient MIU than the mean friction coefficient MIU of the high friction part.
[5] The biological information measuring belt according to above [3],
wherein a skin-side surface of the second fabric includes the high friction part, and
the skin-side surface of the first fabric includes a low friction part having a lower mean friction coefficient MIU than the mean friction coefficient MIU of the high friction part.
[6] The biological information measuring belt according to above [5],
wherein a skin-side surface of the band member includes the low friction part.
[7] The biological information measuring belt according to any one of above [4] to [6],
wherein the low friction part is present in at least a part of a region within 5 cm from an outer edge of the electrode.
[8] The biological information measuring belt according to above [2] or [3],
wherein the skin-side surface of the first fabric includes the high friction part, and
a skin-side surface of the band member includes a low friction part having a lower mean friction coefficient MIU than the mean friction coefficient MIU of the high friction part.
[9] The biological information measuring belt according to above [8],
wherein the high friction part is present in at least a part of a region within 5 cm from an outer edge of the electrode.
[10] The biological information measuring belt according to any one of above [2] to [9],
wherein the band member has a longer length in the body peripheral direction than a length of the first fabric in the body peripheral direction.
[11] The biological information measuring belt according to any one of above [3], [5], and [6],
wherein the second fabric has a shorter length in the body peripheral direction than a length of the first fabric in the body peripheral direction.
[12] The biological information measuring belt according to any one of above [2] to [1], including a moving member including:
a third fabric; and
a belt insertion part into which the band member is inserted, the belt insertion part being provided on a surface opposite to a skin-side surface of the third fabric,
wherein the moving member is movable in the body peripheral direction of the band member, and
the skin-side surface of the third fabric includes the high friction part.
[13] The biological information measuring belt according to any one of above [1] to [12],
wherein the tensile strength at 5% stretching is 0.2 N/cm or more, and
the tensile strength at 10% stretching is 0.8 N/cm or more.
[14] The biological information measuring belt according to any one of above [1] to [13],
wherein the mean friction coefficient MIU of the high friction part is 1.0 or less.
[15] The biological information measuring belt according to any one of above [1] to [14],
wherein the high friction part is a part on which at least one fine fiber having a single fiber diameter of 1,000 nm or less is exposed.
[16] The biological information measuring belt according to above [15],
wherein the single fiber diameter of the fine fiber is less than 1,000 nm.
[17] The biological information measuring belt according to above [15].
wherein the single fiber diameter of the fine fiber is 800 nm or less.
[18] The biological information measuring belt according to any one of above [1] to [17],
wherein the skin-side surface of the first fabric has the high friction part.
[19] The biological information measuring belt according to any one of above [1] to [18],
wherein when an exposed area of the skin-side surface of the biological information measuring belt is defined as 100 area %, an area ratio of the high friction part is 5 area % or more, and 70 area % or less.
[20] The biological information measuring belt according to above [19],
wherein the area ratio of the high friction part is 10 area % or more, and 40 area % or less.
[21] The biological information measuring belt according to any one of above [1] to [20],
wherein when a fabric included in the high friction part is defined as 100 mass %, the high friction part includes 5 mass % or more, and 60 mass % or less of an elastic yarn.
[22] The biological information measuring belt according to any one of above [1] to [20],
wherein when a fabric included in the high friction part is defined as 100 mass %, the high friction part includes 10 mass % or more, and 30 mass % or less of an elastic yarn.
[23] The biological information measuring belt according to any one of above [1] to [22],
wherein the skin-side surface of the first fabric has the low friction part having the lower mean friction coefficient MIU than the mean friction coefficient MIU of the high friction part.
[24] The biological information measuring belt according to above [23].
wherein the skin-side surface of the first fabric further has the high friction part.
[25] The biological information measuring belt according to any one of above [4] to [24],
wherein on the skin-side surface of the biological information measuring belt, an area ratio of the low friction part is larger than the area ratio of the high friction part.
[26] The biological information measuring belt according to any one of above [4] to [25],
wherein the low friction part is a part on which at least one fiber having a single fiber diameter of more than 1 μm is exposed.
[27] The biological information measuring belt according to any one of above [4] to [26],
wherein when the exposed area of the skin-side surface of the biological information measuring belt is defined as 100 area %, the area ratio of the low friction part is 30 area % or more, and 95 area % or less.
[28] The biological information measuring belt according to any one of above [4] to [26],
wherein the area ratio of the low friction part is 60 area % or more, and 90 area % or less.
[29] The biological information measuring belt according to any one of [4] to [28],
wherein a fabric included in the low friction part is defined as 100 mass %, the low friction part includes more than 60 mass %, and 100 mass % or less of an elastic yarn.
[30] The biological information measuring belt according to any one of above [4] to [28],
wherein a fabric included in the low friction part is defined as 100 mass %, the low friction part includes 80 mass % or more, and 100 mass % or less of an elastic yarn.
[31] The biological information measuring belt according to any one of above [1] to [30],
wherein the electrode has a first insulating layer formed on the skin-side surface of the first fabric and an electrically conductive layer formed on the first insulating layer.
[32] The biological information measuring belt according to any one of above [2] to [31],
wherein the band member has a shorter length in a width direction than a length of the first fabric in a width direction.
[33] The biological information measuring belt according to any one of above [3] to [32],
wherein the second fabric has a shorter length in a width direction than the length of the first fabric in the width direction.

### Advantageous Effects of the Invention

According to the present invention, the above configurations can provide the biological information measuring belt fora calf that facilitates measurement of biological information.

### BRIEF DESCRIPTION OF DRAWINGS

FIG. 1 is a plan view of a skin side of a biological information measuring belt for a calf according to a first embodiment.

FIG. 2 is a plan view of a skin side of a biological information measuring belt for a calf according to a second embodiment.

FIG. 3 is a plan view of a skin side of a band member.

FIG. 4 is a plan view of a skin side of a biological information measuring belt for a calf according to a third embodiment.

FIG. 5 is a plan view of a skin side of a biological information measuring belt for a calf according to a forth embodiment.

FIG. 6 is a plan view of a skin side of a moving member.

FIG. 7 is a plan view of a front side opposite to the skin side of the moving member.

### DESCRIPTION OF EMBODIMENTS

A biological information measuring belt for a calf according to an embodiment of the present invention has a first fabric and an electrode provided on a skin-side surface of the first fabric. A skin-side surface of the biological information measuring belt has a part having a mean friction coefficient MIU of 0.40 or more (hereinafter referred to as a high friction part), and the biological information measuring belt has a tensile strength of 2.8 N/cm or less at 5% stretching and has a tensile strength of 4.0 N/cm or less at 10% stretching under following conditions. The conditions are that the biological information measuring belt is held with chucks of a tensile tester in such a manner that a distance between the chucks becomes 45 cm and a center of the first fabric is positioned at a center between the chucks, and then the biological information measuring belt is stretched at a stretching speed of 100 mm/minute.

The skin-side surface of the biological information measuring belt having the high friction part having the mean friction coefficient MIU of 0.40 or more in the above manner makes the biological information measuring belt less likely to be positionally displaced, thus being able to facilitate measurement of biological information. Although a calf rapidly grows and becomes large in body peripheral length on a daily basis, the tensile strengths of the biological information measuring belt within the above ranges enable the belt to be stretched in line with the growth of the calf. As a result, a skin abnormality or the like of the calf that is caused by a pressure of the belt can be reduced.

Hereinafter, the present invention is more specifically described on the basis of the following embodiment. The present invention, however, is not limited by the following embodiment, and can also be absolutely implemented with appropriate changes to the embodiment within a scope in compliance with the intent described above and below, and all the changes are to be encompassed within the technical scope of the present invention. In each of the drawings, the symbols of members, or the like are sometimes omitted for convenience. In such a case, the description and other drawings are to be referred to. Further, the drawings put priority on contributing to understanding of the features of the present invention, and thus the dimensions of various members in the drawings are sometimes different from the actual dimensions thereof.

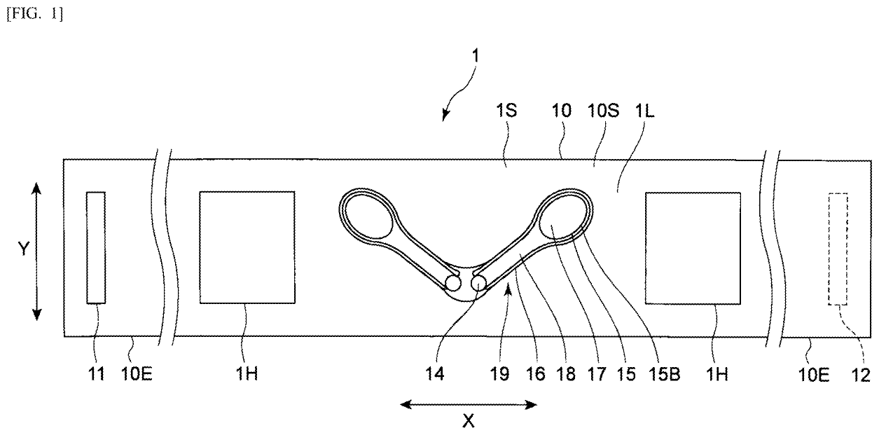

With reference to FIG. 1, a biological information measuring belt for a calf according to a first embodiment is described. FIG. 1 is a plan view of a skin side of the biological information measuring belt according to the first embodiment. In FIG. 1, broken lines represent a member provided on a front side opposite to the skin side.

A biological information measuring belt for a calf 1 according to the first embodiment has a first fabric 10 and an electrode 15 provided on a skin-side surface 10S of the first fabric 10. A skin-side surface 1S of the biological information measuring belt for a calf 1 has a high friction part 1H having a mean friction coefficient MIU of 0.40 or more. The biological information measuring belt for a calf 1 has a tensile strength of 2.8 N/cm or less at 5% stretching and a tensile strength of 4.0 N/cm or less at 10% stretching under following conditions. The conditions are that the biological information measuring belt for a calf 1 is held with chucks of a tensile tester in such a manner that a distance between the chucks becomes 45 cm and a center of the first fabric 10 is positioned at a center between the chucks, and the biological information measuring belt for a calf 1 is stretched at a stretching speed of 100 mm/minute.

The tensile strengths of the biological information measuring belt for a calf 1 within the above ranges enable the biological information measuring belt for a calf 1 to be stretched in line with growth of the calf. As a result, a skin abnormality or hair discoloration of the calf that is caused by a pressure of the biological information measuring belt for a calf 1 can be reduced. In addition, this also enables the biological information measuring belt for a calf 1 to be worn by the calf for such a long period as 2 days or more. For this reason, the tensile strength at 5% stretching is more preferably 2.0 N/cm or less, and the tensile strength at 10% stretching is more preferably 3.0 N/cm or less. Further preferably, the tensile strength at 5% stretching is 1.4 N/cm or less, and the tensile strength at 10% stretching is 2.0 N/cm or less. On the other hand, the tensile strength of 0.2 N/cm or more at 5% stretching and the tensile strength of 0.8 N/cm or more at 10% stretching can enhance tight contact of the electrode 15 with skin. For this reason, the tensile strength at 5% stretching is preferably 0.2 N/cm or more, and the tensile strength at 10% stretching is preferably 0.8 N/cm or more. More preferably, tensile strength at 5% stretching is 0.3 N/cm or more, and the tensile strength at 10% stretching is 0.9 N/cm or more. Further preferably, the tensile strength at 5% stretching is 0.4N/cm or more, and the tensile strength at 10% stretching is 1.0 N/cm or more.

When lengths of the biological information measuring belt for a calf 1 in a width direction Y are not uniform between the chucks in the above tensile test, the tensile strength (N/cm) can be calculated by determining an average width of the belt in the width direction Y between the chucks and then dividing a load (N) by the average width. The average width is an average length of a plurality of regions having different lengths in the width direction Y. This average width can be calculated by determining a sum of a value obtained by multiplying a ratio of each length of the regions in a body peripheral direction X to the distance between the chucks (45 cm) by the length of each region in the width direction Y, as described in detail in EXAMPLES.

The skin-side surface 1S of the biological information measuring belt for a calf 1 having the high friction part 1H having the mean friction coefficient MIU of 0.40 or more makes the biological information measuring belt for a calf 1 less likely to be positionally displaced after the biological information measuring belt for a calf 1 is worn. As a result, measurement of biological information can be facilitated. The high friction part 1H has the mean friction coefficient MIU of 0.40 or more, preferably 0.42 or more, and more preferably 0.44 or more. On other hand, the mean friction coefficient MIU may be 1.0 or less, 0.8 or less, 0.7 or less, or 0.6 or less. The high friction part 1H preferably satisfies the above mean friction coefficient MIU in at least either the body peripheral direction X or the width direction Y and more preferably satisfies the above mean friction coefficient MIU in both the body peripheral direction X and the width direction Y.

The high friction part 1H is preferably a part on which at least one fine fiber is exposed. Preferable examples of the fine fiber include a microfiber and a nanofiber. The fine fiber has a single fiber diameter of preferably 8 μm or less, more preferably 1,000 nm or less, further preferably less than 1,000 nm, even further preferably 800 nm or less, and particularly preferably 750 nm or less. On the other hand, the single fiber diameter may be 100 nm or more, 300 nm or more, or 500 nm or more. The fine fiber is preferably a polyester fiber, a nylon fiber, or the like. An example of the polyester fiber includes a polyester fiber containing polyethylene terephthalate, polytrimethylene terephthalate, polybutylene terephthalate, polylactic acid, stereocomplex polylactic acid, or the like. One kind, or two or more kinds of these may be used. In addition, the fine fiber may be a fiber obtained by dissolving and removing a sea component of a sea-island composite fiber. Specific examples of the fine fiber include NANOFRONT (registered trademark) manufactured by Teijin Fibers Ltd., Toraysee (registered trademark) manufactured by Toray Industries, Inc., and Belima (registered trademark) manufactured by KB SEIREN LTD.

The single fiber diameter of the fine fiber can be determined by a resin embedding method. For example, a fiber bundle of a single yarn that is taken from a fabric is straightened and embedded in a resin and then is cut perpendicularly to its fiber axis to obtain a cut piece. Thereafter, a fiber cross section of the cut piece is photographed with an electron microscope. From a diameter of a single fiber of the fiber bundle in the obtained photograph and a magnification scale of the photograph, a diameter of the fiber cross section is determined, and an arithmetic average value of the determined diameters is calculated at n=20. The calculated average value can be defined as the single fiber diameter.

When an exposed area of the skin-side surface 1S of the biological information measuring belt for a calf 1 is defined as 100 area %, an area ratio of the high friction part 1H is preferably 5 area % or more, and more preferably 10 area % or more. This makes the biological information measuring belt for a calf 1 less likely to be positionally displaced. On the other hand, the area ratio of the high friction part 1H is preferably 70 area % or less, more preferably 60 area % or less, further preferably 50 area % or less, and even further preferably 40 area % or less. This can facilitate adjustment of a position of the biological information measuring belt for a calf 1 when the biological information measuring belt for a calf 1 is worn. When a plurality of high friction parts 1H is present, this area ratio refers to a total area ratio of the plurality of high friction parts 1H.

The high friction part 1H is preferably present in a region that is 1 cm or more away from an outer edge 15B of the electrode 15 at a shortest distance. This can facilitate adjustment of a position of the electrode 15 or the like when the biological information measuring belt for a calf 1 is worn. This shortest distance is more preferably 2 cm or more, and further preferably 3 cm or more. On the other hand, this shortest distance may be 50 cm or less, 30 cm or less, or 20 cm or less.

The high friction part 1H may be present on one part of the skin-side surface 1S, or the high friction parts 1H may be present on two or more parts of the skin-side surface 1S. When the high friction parts 1H are present on two or more parts, each of the high friction parts 1H is preferably present in a point-symmetric position with a center of the first fabric 10 being a center of the position.

In FIG. 1, a length of the high friction part 1H in the width direction Y is shorter than a length from one end to the other end of the biological information measuring belt for a calf 1 in the width direction Y, but these lengths may be the same. That is, the high friction part 1H may be provided across the biological information measuring belt for a calf 1 from one end to the other end thereof in the width direction Y.

The biological information measuring belt for a calf 1 preferably has a low friction part 1L having a lower mean friction coefficient MIU than the mean friction coefficient MIU of the high friction part 1H. This can facilitate adjustment of a position of the biological information measuring belt for a calf 1 when the biological information measuring belt for a calf 1 is worn.

The low friction part 1L has the mean friction coefficient MIU of less than 0.40, preferably 0.38 or less, and more preferably 0.36 or less. This can facilitate slight adjustment of a position of the electrode 15 when the biological information measuring belt for a calf 1 is worn. On the other hand, the lower limit of the mean friction coefficient MIU may be 0.05 or more, or 0.1 or more, but is not particularly limited thereto.

When the exposed area of the skin-side surface 1S of the biological information measuring belt for a calf 1 is defined as 100 area %, an area ratio of the low friction part 1L is preferably 30 area % or more, more preferably 40 area % or more, further preferably 50 area % or more, and even further preferably 60 area % or more. On the other hand, the area ratio of the low friction part 1L is preferably 95 area % or less, more preferably 90 area % or less, and further preferably 85 area % or less. This makes the biological information measuring belt for a calf 1 less likely to be positionally displaced. When a plurality of low friction parts 1L is present, this area ratio refers to a total area ratio of the plurality of low friction parts 1L.

On the skin-side surface 1S of the biological information measuring belt for a calf 1, the area ratio of the low friction part 1L is preferably larger than the area ratio of the high friction part 1H. This enables the biological information measuring belt for a calf 1 to be easily worn.

The low friction part 1L is preferably present in at least a part of a region within 5 cm from the outer edge 15B of the electrode 15 at a shortest distance. This can facilitate slight adjustment of a position of the electrode 15 when the biological information measuring belt for a calf 1 is worn. The low friction part 1L is more preferably present in at least a part of the region within 3 cm from the outer edge 15B of the electrode 15 at the shortest distance and further preferably present in at least a part of the region within 1 cm from the outer edge 15B of the electrode 15 at the shortest distance.

The low friction part 1L is preferably a part on which at least one fiber is exposed. This exposed fiber is preferably a fiber having a single fiber diameter of more than 1 μm. The single fiber diameter is more preferably 5 μm or more, and may be 30 μm or less, or 25 μm or less.

When the exposed area of skin-side surface 1S of the biological information measuring belt for a calf 1 is defined as 100 area %, a part on which at least one fiber is exposed (hereinafter may be referred to as a fiber exposure part) has an area ratio of preferably 60 area % or more, and more preferably 70 area % or more. On the other hand, the area ratio of the fiber exposure part may be 95 area % or less, or 90 area % or less. When a plurality of fiber exposure parts is present, this area ratio refers to a total area ratio of the plurality of fiber exposure parts.

A fabric included in the high friction part 1H preferably includes an elastic yarn and a yarn including the fine fiber. The elastic yarn is a yarn having rubber-like elasticity. The elastic yarn may be a monofilament or a multifilament. Specific examples of the elastic yarn include a polyurethane elastic yarn, a polyester-based elastic yarn, a polyolefin-based elastic yarn, a natural rubber yarn, a synthetic rubber yarn, and a yarn including a stretchable composite fiber. One kind, or two or more kinds of the elastic yarns may be used. Among these, preference is given to the polyurethane elastic yarn because the polyurethane elastic yarn is excellent in yarn elasticity, a heat setting property, chemical resistance, or the like. The polyurethane elastic yarn may be, for example, a fusion bonded-type polyurethane elastic yarn, a bonded-type polyurethane elastic yarn, or the like. A specific example of the elastic yarn includes LYCRA (registered trademark) fiber manufactured by TORAY OPELONTEX CO., LTD.

The elastic yarn has an elongation at break of preferably 100% or more, more preferably 200% or more, and further preferably 400% or more. On the other hand, the elastic yarn may have the elongation at break of 1,000% or less, or 900% or less. This elongation is, for example, defined as 100% when a distance between chucks is doubled after stretching.

The fabric included in the high friction part 1H may include a yarn other than the above elastic yarn (hereinafter referred to as a non-elastic yarn). Inclusion of the non-elastic yarn can prevent the fabric from being excessively stretched. The non-elastic yarn may be a filament yarn or a spun yarn. Specific examples of the non-elastic yarn include a yarn including a multifilament of a synthetic fiber such as polyethylene terephthalate, polytrimethylene terephthalate, nylon 6, nylon 66, aramid, acrylic, acrylate, polyethylene, polypropylene, polyarylate, or polybenzazole; a chemical fiber (a semisynthetic fiber) such as rayon, acetate, lyocell, or cupra; a natural fiber such as cotton, wool, or silk; or a carbon fiber. One kind, or two or more kinds of the non-elastic yarns may be used. Although the non-elastic yarn may be the filament yarn or the spun yarn, preference is given to the filament yarn. The filament yarn is preferably a multifilament yarn, more preferably at least one selected from the group consisting of a polyethylene multifilament yarn, a polyethylene terephthalate multifilament yarn, and a nylon 6 multifilament yarn, and further preferably the polyethylene multifilament yarn.

When the fabric included in the high friction part 1H is defined as 100 mass %, a content of the elastic yarn is preferably 5 mass % or more. This can facilitate reduction in the tensile strength at stretching. The content is more preferably 8 mass % or more, and further preferably 10 mass % or more. On the other hand, the content of the elastic yarn is preferably 60 mass % or less. This can prevent a decrease in frictional force of the fabric that is caused by excessive stretching of the fabric. The content of the elastic yarn is more preferably 40 mass % or less, and further preferably 30 mass % or less.

The fabric included in the high friction part 1H is preferably a woven fabric or a knitted fabric. Examples of the woven fabric include a single woven fabric and a multiple woven fabric, and preference is given to the multiple woven fabric. Among this, more preference is given to a double woven fabric whose back yarn includes the fine fiber.

The knitted fabric is preferably an insert knitted fabric or a pile knitted fabric in which the yarn including the fine fiber is inserted in such a manner that the yarn is exposed on the skin-side surface and can come into contact with skin. The knitted fabric may be a weft knitted fabric or a warp knitted fabric. The weft knitted fabric includes a circular knitted fabric. When the knitted fabric is the weft knitted fabric, the weft knitted fabric is preferably obtained by a plating knitting method in which a plurality of knitting yarns is knitted into a face and a back of the fabric and the yarn including the fine fiber is disposed on one side of the fabric. When the knitted fabric is the circular knitted fabric, the circular knitted fabric is preferably a single knitted fabric with a stitch such as a jersey stitch, a bare jersey stitch, or a welt jersey stitch, and is preferably a double knitted fabric with an interlock stitch (a smooth stitch) or a double knitted fabric at least whose skin-side surface has an all-knit structure. When the knitted fabric is the warp knitted fabric, the warp knitted fabric preferably has a satin net structure formed by raschel-knitting in which the elastic yarn is inserted in a warp direction and in which the yarn including the fine fiber is inserted into a back reed. In order for the yarn including the fine fiber to be disposed on a skin-side surface of the fabric, the yarn including the fine fiber is preferably disposed, for example, in a middle reed or aback reed as an insert structure. In addition, an evasion wrap structure may be used, or the yarn including the fine fiber may be disposed in a sinker loop surface. The evasion wrap structure is a knitting structure in which an inserted yarn lies on a surface of a knitted fabric without being knitted into the fabric, and reference can be made to, for example, the warp knitting complete works published by NIPPON MAYER LTD.

A fabric included in the low friction part 1L preferably includes a yarn including the fiber having the single fiber diameter of more than 1 μm and more preferably includes the yarn including the fiber having the single fiber diameter of more than 1 μm, and an elastic yarn. In addition, the fabric included in the low friction part 1L may include a non-elastic yarn. For the elastic yarn and the non-elastic yarn, reference can be made to the above descriptions of the high friction part 1H.

When the fabric included in the low friction part 1L is defined as 100 mass %, a content of the elastic yarn is preferably more than 60 mass %. This facilitates obtention of the properties in which the tensile strength at 5% stretching is 2.8 N/cm or less, and the tensile strength at 10% stretching is 4.0 N/cm or less. The content is more preferably 70 mass % or more, and further preferably 80 mass % or more. On the other hand, the content of the elastic yarn may be 100 mass % or less, or 90 mass % or less.

Examples of the fabric included in the low friction part 1L include a knitted fabric, a woven fabric, and a non-woven fabric. Among these, preference is given to the knitted fabric because the knitted fabric is excellent in stretchability. Examples of the knitted fabric include a weft knitted fabric and a warp knitted fabric. The weft knitted fabric includes a circular knitted fabric. Examples of the weft knitted fabric (the circular knitted fabric) include fabrics with stitches such as a jersey stitch (a plain stitch), a bare jersey stitch, a welt jersey stitch, a fraise stitch (a rib stitch), a purl stitch, a half tubular stitch, an interlock stitch, a tuck stitch, a float stich, a half cardigan stitch, a lace stitch, a plating stitch, and a jersey stitch. Examples of the warp knitted fabric include fabrics with stitches such as a single denbigh stitch, an open-loop denbigh stitch, a single atlas stitch, a double cord stitch, a half stitch, a half base stitch, a satin stitch, a single tricot stitch, a double tricot stitch, a half tricot stitch, a single raschel stitch, a double raschel stitch, and a jacquard stitch. Examples of the woven fabric include woven fabrics formed by a plain weave, a twill weave, or a satin weave. The woven fabric is not limited to a single woven fabric and may be a multiple woven fabric such as a double woven fabric or a triple woven fabric. These knitted fabric and woven fabric, and the like may be formed in a mesh pattern or be used as a double face fabric in which the two fabrics are stuck together.

As shown in FIG. 1, the biological information measuring belt for a calf 1 preferably has a first joining member 11 and a second joining member 12. Each of the first joining member 11 and the second joining member 12 is more preferably provided at an end part 10E of the first fabric 10 in the body peripheral direction X. Examples of the first joining member 11 and the second joining member 12 include a hook-and-loop fastener such as MAGICTAPE (registered trademark) or FREEMAGIC (registered trademark), a buckle, a hook, and a loop. This enables the biological information measuring belt for a calf 1 to be worn by a calf by joining together both the end parts of the biological information measuring belt for a calf 1 in the body peripheral direction X.

Examples of a shape of the first fabric 10 include a rectangular shape such as a square shape or an oblong shape, a circular shape, and an elliptical shape in plan view. Among these, preference is given to the rectangular shape, and more preference is given to the oblong shape. The first fabric 10 having a longer length in the body peripheral direction X in which tension is applied than a length in the width direction Y facilitates enhancement of tight contact of the electrode 15 with skin.

The electrode 15 is provided on the skin-side surface 10S of the first fabric 10. For example, bringing the electrode 15 into direct or indirect contact with skin of a calf enables acquisition of biological information of the calf. Between the electrode 15 and the skin of the calf, an electrically conductive material or the like may be present. The electrode 15 is preferably positioned in such a manner that at least a part of the electrode 15 faces a body of the calf when the biological information measuring belt for a calf 1 is worn by the calf. This facilitates acquisition of electrocardiographic information of the calf.

Not only one but also two or more electrodes 15 may be provided on the skin-side surface 10S of the first fabric 10. When a plurality of electrodes 15 is provided, the length of the first fabric 10 in the width direction Y is preferably shorter than an average distance between each center of the electrodes 15. This can reduce occurrence of a wrinkle or damage of the first fabric 10 around the electrodes 15.

The electrode 15 preferably has a first insulating layer 16 formed on the skin-side surface 10S of the first fabric 10 and an electrically conductive layer 17 formed on the first insulating layer 16. Between the skin-side surface 10S and the first insulating layer 16, another layer such as an adhesive layer may be present. In addition, between the first insulating layer 16 and the electrically conductive layer 17, another layer such as a hot-melt layer may be present.

The first insulating layer 16 may contain, for example, an insulating resin, and a kind of the resin is not particularly limited. For example, this resin may preferably be a polyurethane-based resin, a silicone-based resin, a vinyl chloride-based resin, an epoxy-based resin, or a polyester elastomer. Among these resins, more preference is given to the polyurethane-based resin that is excellent in adhesiveness to the electrically conductive layer 17. One kind, or two or more kinds of these resins may be used. A method for forming the first insulating layer 16 is not particularly limited, and the first insulating layer 16 can be formed, for example, by dissolving or dispersing the insulating resin in a solvent (preferably water), applying the resulting solution to or performing printing with the resulting solution on release paper or a release film to form a coating film, and volatilizing the solvent contained in the coating film and thus drying the coating film. A commercially available resin sheet or resin film can also be used. The first insulating layer 16 preferably has an average film thickness of 10 to 200 μm.

The electrically conductive layer 17 is a layer for securing electric conduction. The electrically conductive layer 17 preferably contains an electrically conductive filler and a resin. This resin is preferably a stretchable resin.

The stretchable resin is, for example, preferably a urethane resin, natural rubber, synthetic rubber, an elastomer, silicone rubber, fluoro-rubber, or the like, and the stretchable resin at least preferably contains rubber containing a sulfur atom and/or rubber containing a nitrile group. The sulfur atom and the nitrile group have a high affinity for the electrically conductive filler (particularly metal powder), and the rubber has high stretchability and thus can easily avoid the generation of cracks or the like at the time of stretching. Such a resin enables the electrically conductive filler to be easily retained in a uniformly dispersed manner and can reduce a change ratio of electric resistance at the time of stretching. Examples of the rubber containing the sulfur atom include, in addition to rubber containing a sulfur atom, an elastomer containing a sulfur atom. The sulfur atom is contained in a form of a sulfide bond or a disulfide bond in a main chain of a polymer, a mercapto group in a side chain or at a terminal, or the like. Examples of the rubber containing the nitrile group include, in addition to rubber containing a nitrile group, an elastomer containing a nitrile group. Particularly, an acrylonitrile-butadiene copolymer rubber that is a copolymer of butadiene and acrylonitrile is a preferable example. As a commercially available product that can be used as the rubber containing the nitrile group, “Nipol (registered trademark) 1042” manufactured by Zeon Corporation is a preferable example. The stretchable resin contained in the electrically conductive layer 17 may be one kind, or two or more kinds of the resins. In the entire resin contained in the electrically conductive layer 17, a total amount of the rubber containing the sulfur atom and the rubber containing the nitrile group is preferably 95 mass % or more, more preferably 98 mass % or more, and further preferably 99 mass % or more.

The electrically conductive filler may be, for example, metal powder, metal nanoparticles, or an electrically conductive material other than the metal powder. The electrically conductive filler may be one kind, or two or more kinds of the above materials. Examples of the metal powder include noble metal powder such as silver powder, gold powder, platinum powder, or palladium powder; base metal powder such as copper powder, nickel powder, aluminum powder, or brass powder; plated powder obtained by plating different types of particles made of inorganic substances such as a base metal and silica with a noble metal such as silver; and an alloyed base metal powder obtained by alloying a base metal and a noble metal such as silver.

The electrically conductive layer 17 can be formed by using for example, a composition obtained by dissolving or dispersing each component in an organic solvent (hereinafter, may be referred to as an electrically conductive paste). The resin contained in the electrically conductive layer 17 (in other words, a solid content of the stretchable resin in a total solid content of the electrically conductive paste for forming the electrically conductive layer) is preferably 5 to 50 mass %, and more preferably 10 to 40 mass %. On the other hand, the electrically conductive filler in the electrically conductive layer 17 is preferably 50 to 95 mass %, and more preferably 60 to 90 mass %. This configuration enables the electrically conductive layer to easily attain both electric conductivity and stretchability.

The electrically conductive layer 17 can be formed, for example, directly on the first insulating layer 16 by using a composition (the electrically conductive paste) obtained by dissolving or dispersing each component in the organic solvent, or can be formed, for example, by forming a coating film through applying the composition or performing printing with the composition in a desired pattern, and volatilizing the organic solvent contained in the coating film and thus drying the coating film. The electrically conductive layer 17 may also be formed, for example, by applying the electrically conductive paste on or performing printing with the electrically conductive paste on a release sheet or the like to form a coating film, volatilizing the organic solvent contained in the coating film and thus drying the coating film to form a sheet-shaped electrically conductive layer 17 in advance, and laminating the sheet-shaped electrically conductive layer 17 in a desired pattern on the first insulating layer 16.

The electrically conductive layer 17 has a dried film thickness of preferably 10 to 150 μm, more preferably 20 to 130 μm, and further preferably 30 to 100 μm. This enables the electrically conductive layer 17 to attain both durability and wear comfort.

The electrically conductive layer 17 is preferably covered with a second insulating layer 18 except for a part other than the electrode 15 or except for a part other than an electronic device connection part 14 and the electrode 15. The second insulating layer 18 can prevent the electrically conductive layer 17 from coming into contact with water such as rain, snow, or sweat. For a resin contained in the second insulating layer 18, reference can be made to the descriptions of the resin contained in the first insulating layer 16 formed on the skin-side surface of the first fabric 10. The resin contained in the second insulating layer 18 may be the same as or different from the resin contained in the first insulating layer 16 but is preferably the same. Using the same resin can reduce damage on the electrically conductive layer 17 that is caused by unevenly applied stress during stretching or contraction of the electrically conductive layer 17 and the insulating layers. The second insulating layer 18 can be formed by the same forming method as the first insulating layer 16. The second insulating layer 18 preferably has an average film thickness of 10 to 200 μm.

On the skin-side surface 10S of the first fabric 10, a wire 19 is preferably provided. The wire 19 preferably has the first insulating layer 16, the electrically conductive layer 17 formed on the first insulating layer 16, and the second insulating layer 18 formed on the electrically conductive layer 17. Through the wire 19, biological information acquired by the electrode 15 can be transmitted.

Between the skin-side surface 10S and the first insulating layer 16, another layer such as an adhesive layer may be present. In addition, between the first insulating layer 16 and the electrically conductive layer 17, another layer such as a hot-melt layer may be present. Between the electrically conductive layer 17 and the second insulating layer 18, another layer such as a hot-melt layer may be present.

In FIG. 1, the electrode 15 includes the first insulating layer 16 provided on the skin-side surface of the first fabric 10 and the electrically conductive layer 17 provided on the first insulating layer 16 but the electrode 15 may include another layer. The electronic device connection part 14 may include the first insulating layer 16 provided on the skin-side surface of the first fabric 10 and the electrically conductive layer 17 provided on the first insulating layer 16. The electronic device connection part 14 enables an electronic device to be connected to a front side surface opposite to the skin-side surface 10S of the first fabric 10 through a connector such as a snap fastener. In addition, a protective layer may be formed in such a manner that a skin-side surface of the electronic device connection part 14 is covered.

The electrode 15 preferably has an electrically conductive structure, and when the electrically conductive structure has a load of 14.7 N applied thereto in the body peripheral direction X or the width direction Y, the electrically conductive structure preferably has a stretching rate of 3% or more, and 60% or less in at least one of the directions. The stretching rate of 3% or more allows the electrode 15 to sufficiently and easily follow movement of the first fabric 10, thus making the electrode 15 less likely to be peeled from the first fabric 10. For this reason, the stretching rate is more preferably 5% or more, and further preferably 10% or more. On the other hand, the stretching rate of 60% or less can easily prevent measurement accuracy of biological information from being degraded due to excessive stretching of the electrode 15. For this reason, the stretching rate is more preferably 55% or less, and further preferably 50% or less. This stretching rate can be measured by, for example, obtaining a test piece with a prescribed size, mounting the test piece to an Instron tensile tester, and subjecting the test piece to measurement at a speed of 300 mm/minute and a load of 14.7 N.

Examples of the electrode having the electrically conductive structure include a woven fabric, a knitted fabric, and a non-woven fabric made of: an electrically conductive fiber or an electrically conductive yarn obtained by covering a base fiber with an electrically conductive polymer, a fiber whose surface is covered with an electrically conductive metal such as silver, gold, copper, or nickel; an electrically conductive yarn made of an electrically conductive fine metal wire; or an electrically conductive yarn obtained by mixed spinning of an electrically conductive fine metal wire and a non-electrically conductive fiber. In addition, an electrode obtained by embroidering a non-electrically conductive fabric with the electrically conductive yarn can also be used as the electrode having the electrically conductive structure.

The biological information measuring belt for a calf 1 is preferably configured to measure electrocardiographic information. The biological information measuring belt for a calf 1 is less likely to cause positional displacement of the electrode 15 due to the high friction part 1H and thus can be suitably used for measuring the electrocardiographic information.

The biological information measuring belt for a calf 1 preferably includes an electronic device having a function of calculating an electrical signal acquired by the electrode. The electronic device can calculate and process the electrical signal acquired by the electrode. This allows acquisition of biological information such as cardiac potential, a heart rate, a pulse rate, a breathing rate, blood pressure, body temperature, myopotential, or sweating. The electronic device is preferably attachable to and detachable from cloth. In addition, the electronic device preferably includes a display means, a storage means, a communication means, a USB connector, or the like. The electronic device may include, for example, a sensor capable of measuring environmental information such as atmospheric temperature, humidity, or atmospheric pressure, or a sensor capable of measuring positional information with GPS.

The biological information measuring belt for a calf 1 can measure cardiac potential, myopotential, or the like by, for example, including two or more electrodes 15 on the skin-side surface 10S of the first fabric 10. Providing a non-contact electrode on the skin-side surface 1S or on a surface opposite to the skin-side surface 1S of the biological information measuring belt for a calf 1 enables measurement of an impedance change of a body and thus measurement of pulse, breathing, an exercise state, or the like. In this case, a skin-side surface of the electrically conductive layer 17 of the electrode 15 may be covered with the second insulating layer 18 or the like and may not be exposed.

The biological information measuring belt for a calf 1 preferably covers at least a part of a thorax, an abdomen, a back, a front leg, a rear leg, a neck, or a face of the calf and more preferably covers at least a part of the thorax or the abdomen. Examples of cattle that is made to wear the biological information measuring belt for a calf 1 include livestock cattle and a dairy cow, and preference is given to the livestock cattle.

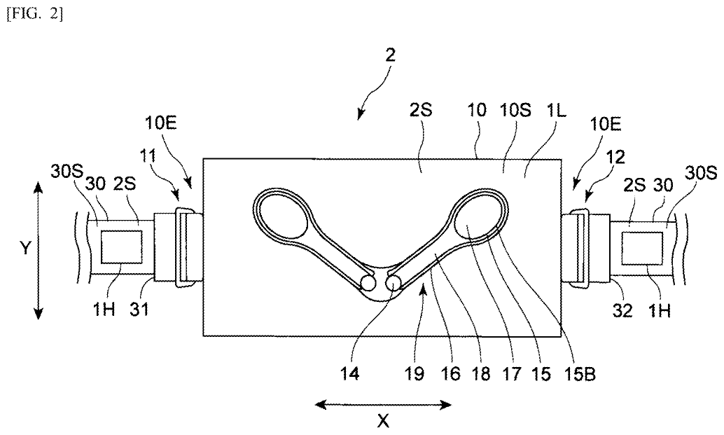

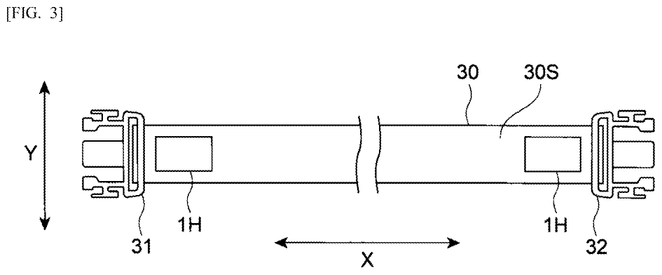

Next, with reference to FIGS. 2 and 3, a biological information measuring belt for a calf 2 according to a second embodiment is described. FIG. 2 is a plan view of a skin side of the biological information measuring belt for a calf 2 of the second embodiment. FIG. 3 is a plan view of a skin side of a band member. In each figure, elements marked with the same reference signs as in FIG. 1 represent the same elements, and reference can be made to the descriptions of the biological information measuring belt for a calf 1 according to the first embodiment.

As shown in FIG. 2, the biological information measuring belt for a calf 2 has a band member 30 provided at an end part 10E of a first fabric 10 in a body peripheral direction X. As shown in FIG. 2, a skin-side surface 2S of the biological information measuring belt for a calf 2 has a high friction part 1H having a mean friction coefficient MIU of 0.40 or more. Specifically, a skin-side surface 30S of the band member 30 has the high friction part 1H. On the other hand, a skin-side surface 10S of the first fabric 10 has a low friction part 1L. In this manner, the high friction part 1H may be provided on a part other than the skin-side surface 10S of the first fabric 10. In addition, the high friction part 1H may be provided on both of the skin-side surface 30S of the band member 30 and the skin-side surface 10S of the first fabric 10. The band member 30 may also have the low friction part 1L on apart other than the high friction part 1H. For preferable properties, fabrics, materials, area ratios of these high friction part 1H and low friction part 1L, preferable distances of the high friction part 1H and the low friction part 1L from an outer edge 15B of an electrode 15, preferable area ratios of fiber exposure parts, or the like, reference can be made to the descriptions of the biological information measuring belt for a calf 1.

When the skin-side surface 10S of the first fabric 10 is defined as 100 area %, an area ratio of the low friction part 1L to the skin-side surface 10S is preferably 50 area % or more, more preferably 60 area % or more, and further preferably 70 area % or more. On the other hand, this area ratio may be 100 area % or less, or 90 area % or less.

When the skin-side surface 30S of the band member 30 is defined as 100 area %, an area ratio of the high friction part 1H to the skin-side surface 30S is preferably 1 area % or more, and more preferably 5 area % or more. On the other hand, this area ratio may be 60 area % or less, 50 area % or less, or 40 area % or less.

As shown in FIG. 2, in the biological information measuring belt for a calf 2, the skin-side surface 30S of the band member 30 preferably has the high friction part 1H, and the skin-side surface 10S of the first fabric 10 preferably has the low friction part 1L having a lower mean friction coefficient MIU than the mean friction coefficient MIU of the high friction part. This can facilitate adjustment of a position of the electrode 15 when the biological information measuring belt for a calf 2 is worn.

As shown in FIG. 3, the band member 30 preferably has a third joining member 31 and a forth joining member 32. Examples of the third joining member 31 and the forth joining member 32 include a female buckle, a male buckle, a hook-and-loop fastener such as MAGICTAPE (registered trademark) or FREEMAGIC (registered trademark), a hook, and a loop. In FIG. 2, the third joining member 31 and the forth joining member 32 are the male buckles and respectively joined to fit inside female buckles of a first joining member 11 and a second joining member 12 provided at the end parts 10E of the first fabric 10 of the biological information measuring belt for a calf 2 in the body peripheral direction X. Connecting the third joining member 31 and the forth joining member 32 to the first joining member 11 and the second joining member 12 in this manner enables the biological information measuring belt for a calf 2 to be worn by a calf.

For the first joining member 11 and the second joining member 12, reference can be made to the descriptions of the biological information measuring belt for a calf 1 according to the first embodiment. In FIG. 2, the first joining member 11 and the second joining member 12 are fastened to the first fabric 10 by inserting fabrics into through holes of the female buckles to form loops and sewing both end parts of the fabrics in longitudinal directions of the fabrics to the end parts 10E of the first fabric 10.

In FIG. 2, the band member 30 is joined to the first fabric 10 through the third joining member 31 and the forth joining member 32, but the joining members may not be employed. In this case, the band member 30 may be fastened to the first fabric 10 through sewing or the like. The band member 30 and the fabric 10 may include a same material or include different materials respectively.

The biological information measuring belt for a calf 2 may have not only one band member 30 but also two or more band members 30. In a case of having two or more band members 30, the band members 30 are preferably joined together in the body peripheral direction X.

The high friction part 1H is preferably present in a region within 20 cm from an end part of the band member 30 on a side of the first fabric 10 in the body peripheral direction X. This can reduce positional displacement of the first fabric 10.

The band member 30 preferably has a longer length in the body peripheral direction X than a length of the first fabric 10 in the body peripheral direction X. The longer length of the band member 30 in the body peripheral direction X enables the biological information measuring belt for a calf 2 to be more easily worn. The length of the band member 30 in the body peripheral direction X is more preferably 1.5 times or more, further preferably 2 times or more, and even further preferably 3 times or more as long as the length of the first fabric 10 in the body peripheral direction X. On the other hand, the length of the band member 30 in the body peripheral direction X may be 30 times or less, or 20 times or less as long as the length of the first fabric 10 in the body peripheral direction X.

The band member 30 preferably has a shorter length in a width direction Y than a length of the first fabric 10 in the width direction Y. This enables the biological information measuring belt for a calf 2 to be easily worn. The length of the band member 30 in the width direction Y is preferably 0.9 times or less, and more preferably 0.8 times or less as long as the length of the first fabric 10 in the width direction Y. On the other hand, the length of the band member 30 in the width direction Y may be 0.1 times or more, or 0.2 times or more as long as the length of the first fabric 10 in the width direction Y.

The length of the first fabric 10 in the width direction Y is preferably 5 to 20 cm and more preferably 7 to 15 cm. On the other hand, the length of the first fabric 10 in the body peripheral direction X is preferably 10 to 60 cm and more preferably 20 to 50 cm.

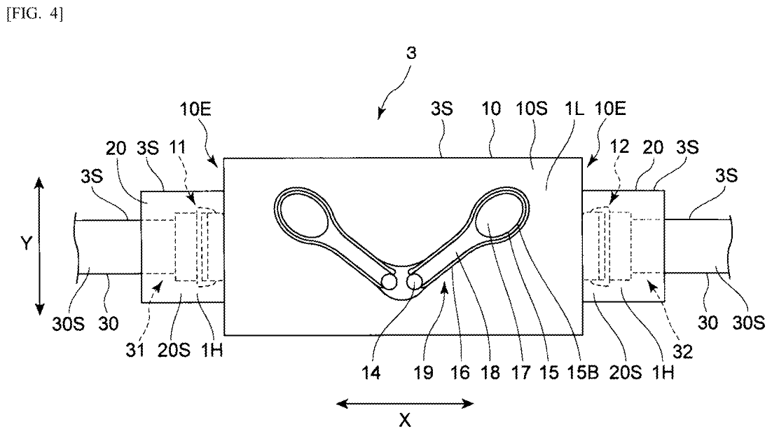

With reference to FIG. 4, a biological information measuring belt for a calf 3 according to a third embodiment is described. In FIG. 4, elements marked with the same reference signs as in FIGS. 1 to 3 represent the same elements, and reference can be made to the descriptions of the biological information measuring belts for a calf 1 and 2 according to the first and the second embodiments.

As shown in FIG. 4, the biological information measuring belt for a calf 3 preferably has a second fabric 20 provided at an end part 10E of a first fabric 10 in a body peripheral direction X and/or on a band member 30 in such a manner that the second fabric 20 is positioned closer to skin than the band member 30. The second fabric 20 is preferably provided at the end part 10E of the first fabric 10 in the body peripheral direction X or on the band member 30. In FIG. 4, one end part of the second fabric 20 in the body peripheral direction X is sewn and fastened to the end part 10E, which makes the second fabric 20 flap-shaped. In addition, the second fabric 20 may be fastened to the band member 30. The second fabric 20 may be provided to the first fabric 10 in a movable manner or on the band member 30 in a movable manner. An example of an aspect of the movable second fabric 20 includes one in which the second fabric 20 is made to form a loop and the band member 30 runs through the loop.

A shown in FIG. 4, a skin-side surface 3S of the biological information measuring belt for a calf 3 has a high friction part 1H having a mean friction coefficient MIU of 0.40 or more. Specifically, a skin-side surface 20S of the second fabric 20 has the high friction part 1H, and a skin-side surface 10S of the first fabric 10 has a low friction part 1L having a lower mean friction coefficient MIU than the mean friction coefficient MIU of the high friction part 1H. This can facilitate slight adjustment of a position of an electrode 15 when the biological information measuring belt for a calf 3 is worn, and this can also reduce positional displacement of the biological information measuring belt for a calf 3.

For a fabric included in the second fabric 20, reference can be made to the fabric included in the high friction part 1H of the biological information measuring belt for a calf 1.

The second fabric 20 preferably has a shorter length in the body peripheral direction X than a length of the first fabric 10 in the body peripheral direction X. This enables the biological information measuring belt for a calf 3 to be easily worn. The length of the second fabric 20 in the body peripheral direction X is preferably 0.8 times or less, and more preferably 0.7 times or less as long as the length of the first fabric 10 in the body peripheral direction X. The length of the second fabric 20 in the body peripheral direction X may be 0.1 times or more, or 0.2 times or more as long as the length of the first fabric 10 in the body peripheral direction X. When a plurality of second fabrics 20 is present, this ratio refers to a ratio of the length of one of the second fabrics 20 in the body peripheral direction X.

The second fabric 20 preferably has a shorter length in a width direction Y than a length of the first fabric 10 in the width direction Y. This enables the biological information measuring belt for a calf 3 to be easily worn. The length of the second fabric 20 in the width direction Y is preferably 0.9 times or less as long as the length of the first fabric 10 in the width direction Y. On the other hand, the length of the second fabric 20 in the width direction Y may be 0.1 times or more, or 0.3 times or more as long as the length of the first fabric 10 in the width direction Y. When a plurality of second fabrics 20 is present, this ratio refers to a ratio of the length of one of the second fabrics 20 in the width direction Y.

A skin-side surface 30S of the band member 30 preferably has the low friction part 1L. This can facilitate adjustment of a position of the band member 30 when the biological information measuring belt for a calf 3 is worn. Though not illustrated in FIG. 4, the skin-side surface 30S of the band member 30 may have the high friction part 1H.

The skin-side surface 10S of the first fabric 10 preferably has the low friction part 1L. This can facilitate adjustment of a position of the first fabric 10 when the biological information measuring belt for a calf 3 is worn.

When the skin-side surface 10S of the first fabric 10 is defined as 100 area %, an area ratio of the low friction part 1L to the skin-side surface 10S is preferably 50 area % or more, more preferably 60 area % or more, and further preferably 70 area % or more. On the other hand, this area ratio may be 100 area % or less, or 90 area % or less.

When the skin-side surface 20S of the second fabric 20 is defined as 100 area %, an area ratio of the high friction part 1H to the skin-side surface 20S is preferably 50 area % or more, more preferably 60 area % or more, and further preferably 70 area % or more. On the other hand, this area ratio may be 100 area % or less, or 90 area % or less.

For preferable properties, fabrics, materials, area ratios of the high friction part 1H and the low friction part 1L, preferable distances of the high friction part 1H and the low friction part 1L from an outer edge 15B of an electrode 15, preferable area ratios of fiber exposure parts, or the like, reference can be made to the descriptions of the biological information measuring belt for a calf 1.

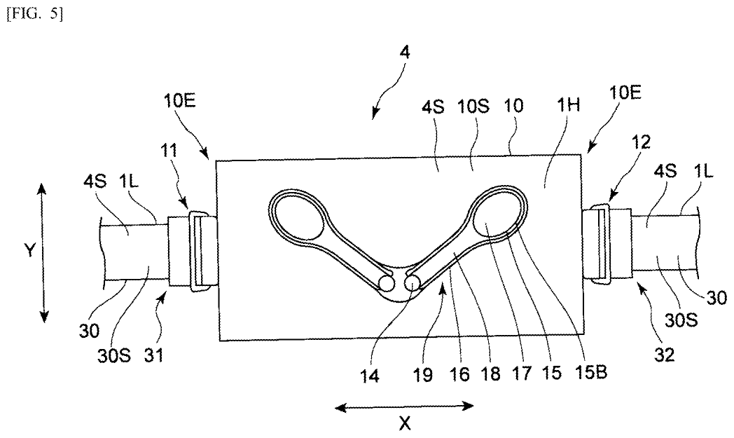

With reference to FIG. 5, a biological information measuring belt for a calf 4 according to a forth embodiment is described. FIG. 5 is a plan view of a skin side of the biological information measuring belt for a calf 4 according to the forth embodiment. In FIG. 5, elements marked with the same reference signs as in FIG. 1 represent the same elements, and reference can be made to the descriptions of the biological information measuring belt for a calf 1 according to the first embodiment.

In the biological information measuring belt for a calf 4, a skin-side surface 10S of a first fabric 10 may have a high friction part 1H, and a skin-side surface 30S of a band member 30 may have a low friction part 1L having a lower mean friction coefficient MIU than a mean friction coefficient MIU of the high friction part 1H. This can facilitate reduction of positional displacement of an electrode 15 after the biological information measuring belt for a calf 4 is worn, and this can also facilitate adjustment of a position of the band member 30.

When the skin-side surface 10S of the first fabric 10 is defined as 100 area %, an area ratio of the high friction part 1H to the skin-side surface 10S is preferably 50 area % or more, and more preferably 70 area % or more. On the other hand, this area ratio may be 100 area % or less, or 90 area % or less.

When the skin-side surface 30S of the band member 30 is defined as 100 area %, an area ratio of the low friction part 1L to the skin-side surface 30S is preferably 50 area % or more, and more preferably 70 area % or more. On the other hand, this area ratio may be 100 area % or less, or 90 area % or less.

The high friction part 1H is preferably present in at least a part of a region within 5 cm from an outer edge 15B of the electrode 15 at a shortest distance. This can reduce positional displacement of the electrode 15 after the biological information measuring belt fora calf 4 is worn. The high friction part 1H is more preferably present in at least a part of the region within 3 cm from the outer edge 15B of the electrode 15 at the shortest distance.

The low friction part 1L is preferably present in a region that is 1 cm or more away from the outer edge 15B of the electrode 15 at a shortest distance. This can facilitate adjustment of a position of the band member 30 or the like when the biological information measuring belt for a calf 4 is worn. This shortest distance is more preferably 2 cm or more, and further preferably 3 cm or more. On the other hand, this shortest distance may be 50 cm or less, 30 cm or less, or 20 cm or less.

Though not illustrated, the biological information measuring belt for a calf 4 may have the second fabric 20 of the biological information measuring belt for a calf 3.

For preferable properties, fabrics, materials, and area ratios of the high friction part 1H and the low friction part 1L, preferable area ratios of fiber exposure parts, or the like, reference can be made to the descriptions of the biological information measuring belt for a calf 1.

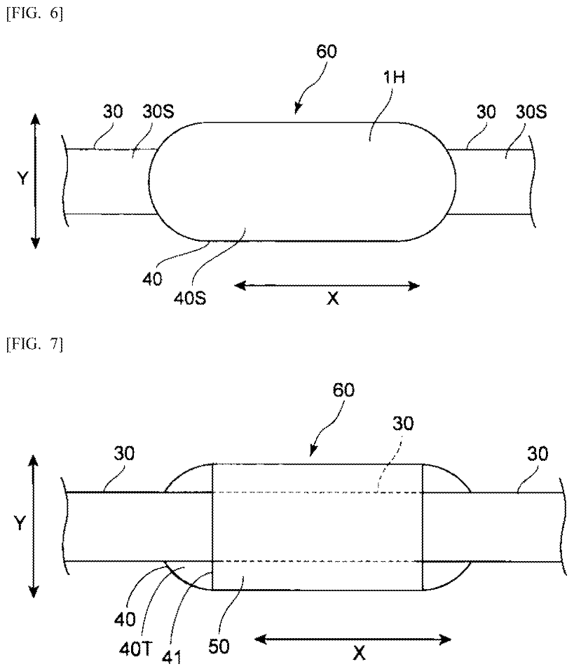

Each of the above biological information measuring belts for a calf 2, 3, and 4 may have a moving member 60 as shown in FIGS. 6 and 7. The moving member 60 has a third fabric 40 and a belt insertion part 41 into which the band member 30 is inserted and that is provided on a surface 40T opposite to a skin-side surface 40S of the third fabric 40. In addition, the moving member 60 is movable in the body peripheral direction X of the band member 30, and the skin-side surface 40S of the third fabric 40 has the high friction part 1H. The moving member 60 movable in the body peripheral direction X in this manner enables adjustment of a position of the high friction part 1H.

The belt insertion part 41 in FIG. 7 includes a forth fabric 50 whose sides along the body peripheral direction X are sewn to the surface 40T of the third fabric 40. The belt insertion part 41 may include, for example, a loop-shaped member.

Examples of a shape of the third fabric 40 include a rectangular shape such as a square shape or an oblong shape, a circular shape, and an elliptical shape in plan view. Among these, preference is given to the oblong shape or the elliptical shape.

For a fabric included in the third fabric 40, reference can be made to the descriptions of the second fabric 20. For a fabric included in the forth fabric 50, reference can be made to the descriptions of the fabric included in the low friction part 1L of the biological information measuring belt for a calf 1.

The present application claims priority based on Japanese Patent Application No. 2020-172036 filed on Oct. 12, 2020. All the contents described in Japanese Patent Application No. 2020-172036 filed on Oct. 12, 2020 are incorporated herein by reference.

### EXAMPLES

Hereinafter, the present invention is more specifically described by way of examples. The present invention, however, is not limited by the following examples, and can also be implemented with changes to the examples within a scope in compliance with the intent described above and below, and all the changes are to be encompassed within the technical scope of the present invention.

### Example 1

A fabric having a same shape as that of a first fabric 10 in FIG. 4 was prepared. First, a bonding double face fabric (W220/2028A) manufactured by MASUMITETU Inc. that had a jersey surface and a mesh surface was cut into an oblong shape of 28 cm×10 cm. Next, an electrode was provided on the jersey surface (a skin-side surface), details of which are as follows.

As a resin, 20 parts by mass of nitrile rubber (Nipol DN003 manufactured by ZEON CORPORATION) were dissolved in 80 parts by mass of isophorone to prepare an NBR solution. In 100 parts by mass of this NBR solution were blended 110 parts of silver particles (“agglomerate silver powder G-35” manufactured by DOWA Electronics Materials Co., Ltd., an average particle diameter: 5.9 μm), and the mixture was kneaded with a triple roll mill to obtain a stretchable silver paste.

Next, the stretchable silver paste was applied onto a release sheet and dried with a hot air dry oven at 120° C. for 30 minutes or longer to prepare a release sheet-attached sheet-shaped electrically conductive layer. An electrically conductive layer of this release sheet-attached sheet-shaped electrically conductive layer corresponds to the above electrically conductive layer 17.

Next, onto a surface of the electrically conductive layer of the release sheet-attached sheet-shaped electrically conductive layer, a polyurethane hot-melt sheet was attached. Thereafter, the release film was peeled to obtain a polyurethane hot-melt sheet-attached sheet-shaped electrically conductive layer. The above polyurethane hot-melt sheet was laminated by using a hot pressing machine under conditions in which a pressure was 0.5 kg/cm², a temperature was 130° C. and a pressing time was 20 seconds.

Next, on a polyurethane hot-melt sheet having a length of 13 cm, the polyurethane hot-melt sheet side of the polyurethane hot-melt sheet-attached sheet-shaped electrically conductive layer having a length of 12 cm was laminated to prepare a laminated body including the polyurethane hot-melt sheet and the sheet-shaped electrically conductive layer. The polyurethane hot-melt sheet corresponds to the above first insulating layer 16.

Next, the first insulating layer 16 and the electrically conductive layer 17 were covered with the same polyurethane hot-melt sheet as one used to prepare the first insulating layer 16 in such a manner that at one end part of the electrically conductive layer 17 in a longitudinal direction of the electrically conductive layer 17, a part of a skin-side surface of the electrically conductive layer 17 was exposed in an elliptical shape (a major axis of 4 cm, a minor axis of 3 cm) and in such a manner that at the other end part, a part of the skin-side surface of the electrically conductive layer 17 was exposed in a circular shape (a diameter of 1 cm). As a result, a second insulating layer 18 was formed. At this time, the first insulating layer 16 and the electrically conductive layer 17 were covered in such a manner that an outer edge of the second insulating layer 18 was to be more outwardly positioned than outer edges of the first insulating layer 16 and the electrically conductive layer 17. In these manners, an electrode wiring sheet having an electronic device connection part 14 having a laminated structure of the first insulating layer 16/the electrically conductive layer 17 in which the electrically conductive layer 17 was exposed; a wire 19 having a laminated structure of the first insulating layer 16/the electrically conductive layer 17/the second insulating layer 18; and an electrode 15 having a laminated structure of the first insulating layer 16/the electrically conductive layer 17 in which the electrically conductive layer 17 was exposed was prepared.

Next, two electrode wiring sheets were symmetrically attached in predetermined positions on the jersey side (the skin-side surface) of the first fabric 10 as shown in FIG. 4. In addition, to a front side of the electronic device connection part 14, an electrocardiographic measurement device, myBeat manufactured by UNION TOOL Co. was connected through a snap button as a connector to obtain the first fabric 10 with electrode wiring.

Next, Grosgrain 7000 manufactured by INOUE RIBBON INDUSTRY Co., Ltd. that was made of polyester and had a width of 5 cm and a length of 6 cm was inserted into a through hole of a female buckle to form a loop, and both end parts of the loop in a longitudinal direction of the loop were sewn to one end part 10E of the first fabric 10 with the electrode wiring to form a first joining member 11 as shown in FIG. 4. At the other end part 10E, a second joining member 12 was formed in a same manner. In addition, to one end part 10E of the first fabric 10 in the body peripheral direction X, an end part of High Grip Tape 1760 manufactured by INOUE RIBBON INDUSTRY Co., Ltd. that included a polyester fiber and a polyurethane fiber and had a width of 6.3 cm and a length of 10 cm in a longitudinal direction of the High Grip Tape 1760 was sewn and fastened in order to form a second fabric 20 having a flap shape in such a manner that the second fabric 20 was positioned closer to skin than the first joining member 11 and the second joining member 12. At the other end part 10E, the second fabric 20 was formed in a same manner. With Binder Tape W1250 manufactured by Asakura Senpu Co., Ltd., outer edges of the first fabric 10 and the second fabric 20 were respectively piped. Also, male buckles were attached to both end parts of Plain Elastic 1100 manufactured by INOUE RIBBON INDUSTRY Co., Ltd. having a width of 5 cm and a length of 90 cm and containing polyester and polyurethane to obtain a band member 30 having a third joining member 31 and a forth joining member 32. The third joining member 31 and the forth joining member 32 of the band member 30 were respectively joined to the first joining member 11 and the second joining member 12 of the first fabric 10 to obtain a biological information measuring belt for a calf 3.

### (Tensile Test)

With TENSILON Universal Material Testing Instrument (model RTF-1310) manufactured by A&D Company, Limited, the biological information measuring belt for a calf 3 was subjected to a tensile test. Specifically, the biological information measuring belt for a calf 3 was first held with chucks of this tester that had a width of 10 cm in such a manner that a distance between the chucks became 45 cm and a center of the first fabric 10 was positioned at a center between the chucks. Next, the biological information measuring belt for a calf 3 was stretched in the body peripheral direction X under conditions that a load cell was 1 kN and a stretching speed was 100 mm/minute. Thereafter, loads (N) at 2.25 cm stretching (5% stretching) and at 4.5 cm stretching (10% stretching) were respectively determined. The loads (N) were divided by an average width of the biological information measuring belt for a calf 3 between the above chucks to determine a tensile strength (N/cm) at 5% stretching and a tensile strength (N/cm) at 10% stretching respectively. The average width between the chucks was calculated according to the following equation. This average width was 7.78 cm. The above tensile test was performed without holding the flap-shaped second fabric 20. That is, the second fabric 20 to which a stress in the body peripheral direction X was not applied was not added as an element to the following equation of the average width.

Average Width=C*A/45+D*B/45

where A represents a length (25 cm) of the first fabric 10 between the chucks in the body peripheral direction X; B represents a total length (20 cm) of the first joining member 11, the second joining member 12, and the band member 30 between the chucks in the body peripheral direction X; C represents a width (10 cm) of the first fabric 10 between the chucks; and D represents a width (5 cm) of the first joining member 11, the second joining member 12, and the band member 30 between the chucks.

### (Friction Test)

With Friction Tester KES-SE manufactured by KATO TECH CO., LTD., a friction block of the tester was swept on a skin-side surface 10S of the first fabric 10 and a skin-side surface 20S of the second fabric 20 while a load was applied to the friction block. Thereafter, a mean friction coefficient (MIU) in the body peripheral direction X, a mean friction coefficient (MIU) in a width direction Y, and a mean friction coefficient (MIU) in the body peripheral direction X and the width direction Y were respectively calculated.

Friction Block: 10 mm-square piano-wire sensor

Specimen Moving Speed: 1 mm/sec

Load: 25 gf

Sensitivity: H

Results of the above tensile test and the friction test are shown in Table 1.

TABLE 1

| | First Fabric | | Second Fabric | | | |
|---|---|---|---|---|---|---|
| | Mean Friction Coefficient | | Mean Friction Coefficient | | Tensile Strength | |
| | (MIU) | | (MIU) | | 5% | 10% |
| | Body Peripheral | Width | Body Peripheral | Width | Stretching | Stretching |
| | Direction | Direction | Direction | Direction | N/cm | N/cm |
| Example 1 | 0.26 | 0.36 | 0.45 | 0.46 | 1.01 | 1.65 |

### (Wearing Test)

A 40-day old calf with black body hair was made to wear the biological information measuring belt for a calf 3 and subjected to electrocardiographic measurement for 2 days. As a result, the biological information measuring belt for a calf 3 gave a stable measurement result with little positional displacement. In addition, no abnormalities were observed in skin and the body hair of the calf.

### REFERENCE SIGNS LIST

1, 2, 3, 4 biological information measuring belt
1S, 2S, 3S, 4S skin-side surface of the biological information measuring belt
1H high friction part
1L low friction part
10 first fabric
10E end part of the first fabric in body peripheral direction
10S skin-side surface of the first fabric
11 first joining member
12 second joining member
14 electronic device connection part
15 electrode
15B outer edge of the electrode
16 first insulating layer
17 electrically conductive layer
18 second insulating layer
19 wire
20 second fabric
20S skin-side surface of the second fabric
30 band member
30S skin-side surface of the band member
31 third joining member
32 forth joining member
40 third fabric
40S skin-side surface of the third fabric
40T surface of the third fabric
41 belt insertion part
50 forth fabric
60 moving member

1. A biological information measuring belt for a calf, the biological information measuring belt comprising:
a first fabric; and
an electrode provided on a skin-side surface of the first fabric,
wherein a skin-side surface of the biological information measuring belt includes a high friction part having a mean friction coefficient MIU of 0.40 or more,
the biological information measuring belt has a tensile strength of 2.8 N/cm or less at 5% stretching and has a tensile strength of 4.0 N/cm or less at 10% stretching under following conditions, and
the conditions are that the biological information measuring belt is held with chucks of a tensile tester in such a manner that a distance between the chucks becomes 45 cm and a center of the first fabric is positioned at a center between the chucks, and then the biological information measuring belt is stretched at a stretching speed of 100 mm/minute.

2. The biological information measuring belt according to claim 1, comprising a band member provided at at least one end part of the first fabric in a body peripheral direction.

3. The biological information measuring belt according to claim 2, comprising a second fabric provided at at least one end part of the first fabric in the body peripheral direction and/or on the band member in such a manner that the second fabric is positioned closer to skin than the band member.

4. The biological information measuring belt according to claim 2,
wherein a skin-side surface of the band member includes the high friction part, and
the skin-side surface of the first fabric includes a low friction part having a lower mean friction coefficient MIU than the mean friction coefficient MIU of the high friction part.

5. The biological information measuring belt according to claim 3,
wherein a skin-side surface of the second fabric includes the high friction part, and
the skin-side surface of the first fabric includes a low friction part having a lower mean friction coefficient MIU than the mean friction coefficient MIU of the high friction part.

6. The biological information measuring belt according to claim 5,
wherein a skin-side surface of the band member includes a low friction part having a lower mean friction coefficient MIU than the mean friction coefficient MIU of the high friction M.

7. The biological information measuring belt according to claim 4,
wherein the low friction part is present in at least a part of a region within 5 cm from an outer edge of the electrode.

8. The biological information measuring belt according to claim 2,
wherein the skin-side surface of the first fabric includes the high friction part, and
a skin-side surface of the band member includes a low friction part having a lower mean friction coefficient MIU than the mean friction coefficient MIU of the high friction part.

9. The biological information measuring belt according to claim 8,
wherein the high friction part is present in at least a part of a region within 5 cm from an outer edge of the electrode.

10. The biological information measuring belt according to claim 2,
wherein the band member has a longer length in the body peripheral direction than a length of the first fabric in the body peripheral direction.

11. The biological information measuring belt according to claim 3,
wherein the second fabric has a shorter length in the body peripheral direction than a length of the first fabric in the body peripheral direction.

12. The biological information measuring belt according to claim 2, comprising a moving member comprising:
a third fabric; and
a belt insertion part into which the band member is inserted, the belt insertion part being provided on a surface opposite to a skin-side surface of the third fabric,
wherein the moving member is movable in the body peripheral direction of the band member, and
the skin-side surface of the third fabric includes the high friction part.
